(19) United States
(12) Patent Application Publication
(10) Pub. No.: US 2010/0228036 A1
(43) Pub. Date: Sep. 9, 2010

(54) PROCESS FOR MAKING GLUCOCORTICOID RECEPTOR LIGANDS

(76) Inventors: Qinghao Chen, Edison, NJ (US); Shinji Fujimori, Rahway, NJ (US); Jacob M. Janey, New York, NY (US); John Limanto, Rahway, NJ (US); Rafik Naccache, Montreal (CA); Andrew F. Nolting, Trenton, NJ (US); Zhigou J. Song, Edison, NJ (US); Neil Strotman, Monmouth Junction, NJ (US); Lushi Tan, Edison, NJ (US); Mark Weisel, Stewartsville, NJ (US)

Correspondence Address: MERCK, P O BOX 2000, RAHWAY, NJ, 07065-0907, US

(21) Appl. No.: 12/739,483

(22) PCT Filed: Oct. 20, 2008
(86) PCT No.: PCT/US08/11935

§ 371 Date: Apr. 23, 2010

(30) Foreign Application Priority Data
Oct. 23, 2007 (US) 60999962

(51) Int. Cl. C07D 231/54

(52) U.S. Cl. 548/359.1

(57) ABSTRACT

The invention encompasses a process for making 2-[1-phenyl-5-hydroxy-4alpha-methyl-hexahydrocyclopenta[f]indazol-5-yl]ethyl phenyl derivatives, which are glucocorticoid receptor ligands, useful for the treatment of inflammatory and immunological diseases.

### CROSS-REFERENCE TO RELATED APPLICATION

This application claims priority from U.S. Provisional Application No. 60/999,962, filed Oct. 23, 2007.

### BACKGROUND OF THE INVENTION

This invention relates to a process for making 2-[1-phenyl-5-hydroxy-4alpha-methyl-hexahydrocyclopenta[f]indazol-5-yl]ethyl phenyl derivatives, which are glucocorticoid receptor ligands useful for the treatment of inflammatory and immunological diseases and believed to have reduced adverse side effects over currently used oral glucocorticoids. These compounds are described in U.S. Nos. 60/853,655, filed Oct. 23, 2006, and 60/923,337, filed Apr. 13, 2007, both of which are hereby incorporated by reference in their entirety.

Intracellular receptors (IR's) are a class of structurally related proteins involved in the regulation of gene expression. The steroid hormone receptors are a subset of this superfamily whose natural ligands are typically comprised of endogenous steroids such as estradiol, progesterone, and cortisol. Man-made ligands to these receptors play an important role in human health and, of these receptors, the glucocorticoid receptor has an essential role in regulating human physiology and immune response. Steroids that interact with the glucocorticoid receptor have been shown to be potent anti-inflammatory agents, although cross-reactivity with other steroid hormone receptors such as the mineralocorticoid, progesterone and androgen receptors can lead to problematic ancillary pharmacology.

The dissociation of transactivation from transrepression at the glucocorticoid receptor is believed to be an approach toward improving the side-effect profile related to steroid therapy. The beneficial anti-inflammatory activity of GR modulators, such as steroids, is believed to occur through the transrepression of genes encoding for proinflammatory cytokines, adhesion molecules and enzymes. Many of the undesirable side-effects associated with such agents are believed to occur through the transactivation, or induction, of gene transcription leading to the downstream perturbation of homeostatic endocrine function. Some of these affected metabolic processes include induced gluconeogenesis, induced amino acid degradation, osteoporosis, suppression of HPA axis, induction of secondary adrenal suppression, changes in electrolyte concentration, changes in lipid metabolism, growth retardation, impaired wound healing and skin thinning. Weak, partial and full agonism of GR related to transrepression and transactivation, including potential antagonism of the receptor regarding transactivation, may be applied to the treatment of inflammatory and autoimmune diseases such as rheumatoid arthritis and asthma. For recent reviews see: (a) Recent Advances in Glucocorticoid Receptor Action; Cato, A. C. B., Schacke, H., Asadullah, K., Eds.; Springer-Verlag: Berlin-Heidelberg, Germany, 2002. (b) Coghlan, M. J.; Elmore, S. W.; Kym, P. R.; Kort, M. E. In Annual Reports in Medicinal Chemistry; Doherty, A. M., Hagmann, W. K., Eds.; Academic Press: San Diego, Calif., USA, 2002; Vol. 37, Ch. 17, pp 167-176.

The present invention describes an efficient and economical process for the preparation of 2-[1-phenyl-5-hydroxy-4alpha-methyl-hexahydrocyclopenta[f]indazol-5-yl]ethyl phenyl derivatives.

### SUMMARY OF THE INVENTION

The invention encompasses a process for making 2-[1-phenyl-5-hydroxy-4alpha-methyl-hexahydrocyclopenta[f]indazol-5-yl]ethyl phenyl derivatives, which are glucocorticoid receptor ligands, useful for the treatment of inflammatory and immunological diseases.

### BRIEF DESCRIPTION OF THE DRAWINGS

FIG. 1. This FIGURE shows the x-ray powder diffraction pattern (XRPD) of the hemihydrate of the compound of Formula Ia.

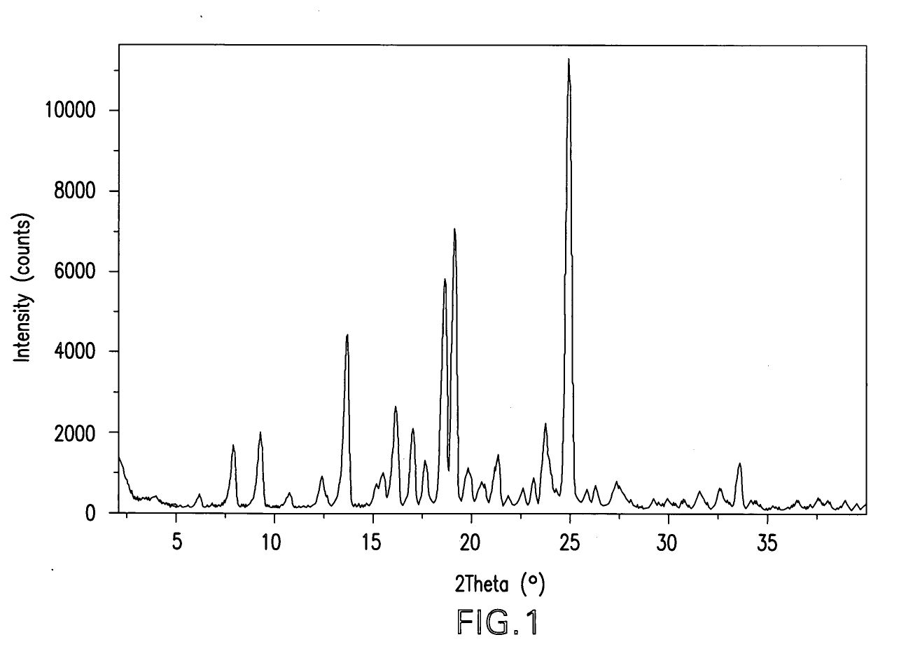

### DETAILED DESCRIPTION OF THE INVENTION

The invention encompasses a process for synthesizing a compound of Formula I

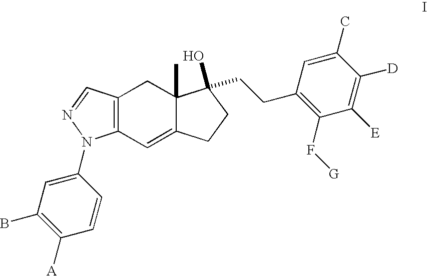

or a pharmaceutically acceptable salt thereof, wherein:
A and B are independently selected from the group consisting of: H, F and Cl;
C, D and E are independently selected from the group consisting of: H, F, Cl, —CN, —CH₃, —OCH₃, phenyl and —CF₃;
F is selected from the group consisting of: a bond, —C(R¹)(R²)— and —C(R¹)(R²)—C(R³)(R⁴)—;
G is selected from the group consisting of: —CN, —OH, —O—C(O)—N(R)(R), —O—C(O)—O—R, —C(O)—R, —C(O)—O—R, —NRR, aryl, substituted aryl, heteroaryl, substituted heteroaryl, —C(R^{a})(R^{b})—N(R)(R), —C(O)—N(R)(R), —C(O)—N(R)—C(R^{a})(R^{b})—R, —C(O)—N(R)—C(R^{a})(R^{b})—C(O)—OR, —C(O)—N(R)—C(R^{a})(R^{b})—C(O)—NRR, —N(R)—C(O)—R, —N(R)—C(O)—OR, —N(R)—C(O)—N(R)(R), —N(R)—S(O)ₙ—X, —S(O)n-N(R)(R), —N(R)—S(O)ₙ—N(R)(R) and —S(O)ₙ—X, wherein n is 0, 1 or 2;
each R is independently selected from the group consisting of: H, C₁₋₈alkyl, haloC₁₋₈alkyl, C₂₋₈alkenyl, haloC₂₋₈alkenyl, C₁₋₈alkoxy and C₃₋₆cycloalkyl-C₁₋₄alkyl-, and
two R groups attached to the same nitrogen atom can be joined together with the nitrogen atom to which they are attached to form a 3- to 7-membered monocyclic ring, said ring optionally substituted with oxo and said ring further optionally substituted with 1 to 3 substituents independently selected from the group consisting of: halo, hydroxyl, C₁₋₄alkyl and C₁₋₄alkoxy;
X is selected from the group consisting of: H, C₁₋₈alkyl, haloC₁₋₈alkyl, C₂₋₈alkenyl, haloC₂₋₈alkenyl, C₁₋₈alkoxy, C₃₋₆cycloalkyl, C₃₋₆cycloalkyl-C₁₋₄alkyl-, —CH₂—S(O)ₖ—CH₃, wherein k is 0, 1 or 2, aryl, substituted aryl, heteroaryl and substituted heteroaryl;
R¹, R², R³ and R⁴ are independently selected from the group consisting of: H, halo, C₁₋₄alkyl, hydroxy, C₃₋₆cycloalkyl and C₁₋₄haloalkyl, and R¹ and R² may be joined together with the carbon atom to which they are attached to form a 3- to 6-membered mono-cyclic ring;
R^{a} and R^{b} are independently selected from the group consisting of: H, C₁₋₄alkyl, C₁₋₄haloalkyl and hydroxy or R^{a} and R^{b} may be joined together with the carbon atom to which they are attached to form a 3- to 6-membered mono-cyclic ring; and
substituted aryl and substituted heteroaryl mean aryl and heteroaryl respectively, each substituted with one to three substituents independently selected from the group consisting of: halo, C₁₋₄alkyl, C₁₋₄-haloalkyl and —CN;
comprising:
(a1) coupling a compound of Formula 4

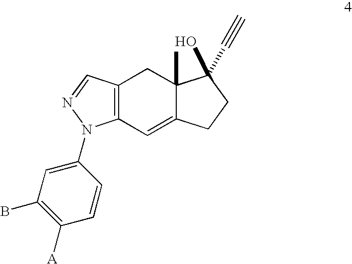

with a compound of Formula 5

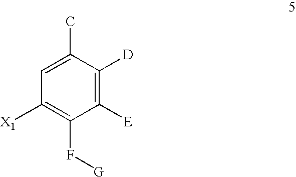

wherein X₁ is selected from the group consisting of: I, Br, Cl and OTf, in the presence of a palladium catalyst, a phosphine ligand and an amine base in a polar aprotic solvent at a first elevated temperature to yield a compound of Formula 6

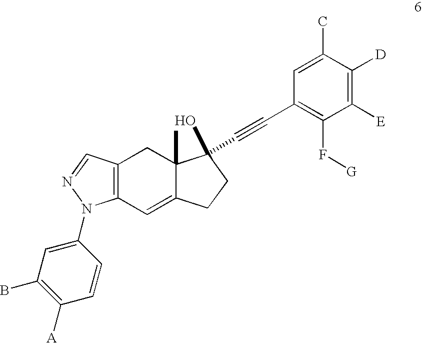

and (a2) hydrogenating the compound of Formula 6 with H₂ in the presence of a metal catalyst to yield the compound of Formula I;
and optionally converting the compound of Formula I into a pharmaceutically acceptable salt.

In an embodiment, the invention encompasses the process of steps (a1) to (a2) above wherein the palladium catalyst is selected from the group consisting of [(allyl)PdCl]₂, palladium hydride, palladium on carbon, palladium(II) acetate, palladium(II) chloride, palladium (II) chloride acetonitrile complex, palladium (II) chloride benzonitrile complex, palladium(II) cyanide, palladium(II) nitrate, palladium(II) oxide, tetrakis(triphenylphosphine)palladium(0), tris(dibenzylideneacetone)dipalladium(0), and organopalladium complexes bearing phosphine ligands.

In another embodiment, the invention encompasses the process of steps (a1) to (a2) above wherein the phosphine ligand is selected from the group consisting of: (t-Bu)₃P.HBF₄, tri-tertbutylphosphine, triphenyl phosphine, tri-ortho-tolylphosphine, tricyclohexylphosphine, diphenylphosphinoferrocene, diphenylphosphinobutane, diphenylphosphinoethane, diphenylphosphinopropane, diphenylphosphinomethane and di-tBu-2-(N-phenylpyrrole)phosphine.

In another embodiment, the invention encompasses the process of steps (a1) to (a2) above wherein the amine base is selected from the group consisting of: N,N-Diisopropylethylamine, diethylamine, triethylamine, diisopropylamine and piperidine.

In another embodiment, the invention encompasses the process of steps (a1) to (a2) above wherein the polar aprotic solvent is selected from the group consisting of: 1,4-Dioxane, tetrahydrofuran, 2-methyltetrahydrofuran, acetone, acetonitrile, dimethylformamide, dimethyl sulfoxide, dimethoxymethane and 2-methyltetrahydrofuran.

In another embodiment, the invention encompasses the process of steps (a1) to (a2) above wherein the first elevated temperature is about 80° C.

In another embodiment, the invention encompasses the process of steps (a1) to (a2) above wherein the metal catalyst is selected from the group consisting of: palladium on carbon, palladium hydroxide on carbon, palladium/platinum amalgam, rhodium on carbon, rhodium on alumina and platinum on carbon.

In another embodiment, the invention encompasses the process of steps (a1) to (a2) above further comprising making the compound of Formula 4 by

(a3) reacting a compound of Formula 2a

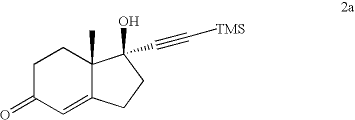

with H—C(O)—O—R, wherein R is C₁₋₄alkyl, in the presence of LiOtBu in a first organic solvent at a first low temperature to yield a compound of Formula 2b

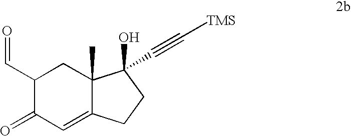

and (a4) quenching the reaction with an organic acid and, without further isolation, reacting the compound of Formula 2b with a compound of Formula 2c

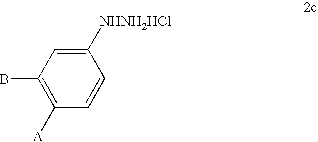

at a second elevated temperature and desilylating with a base, in either order, to yield a compound of Formula 4.

In another embodiment, the invention encompasses the process of steps (a1) to (a4) above wherein the first organic solvent is selected from the group consisting of: tetrahydrofuran, 2-methyltetrahydrofuran, hexane, benzene, toluene, diethyl ether, chloroform, ethyl acetate and dichloromethane.

In another embodiment, the invention encompasses the process of steps (a1) to (a4) above wherein the first low temperature is about 5° C. to about 10° C.

In another embodiment, the invention encompasses the process of steps (a1) to (a4) above wherein the organic acid is selected from the group consisting of: acetic acid, formic acid, benzoic acid and p-toluenesulfonic acid.

In another embodiment, the invention encompasses the process of steps (a1) to (a4) above wherein the second elevated temperature is about 60° C.

In another embodiment, the invention encompasses the process of steps (a1) to (a2) above wherein the base is sodium hydroxide.

In another embodiment, the invention encompasses the process of steps (a1) to (a4) above further comprising making the compound of Formula 2a by (a5) reacting TMS-acetylene-MgCl with CeCl₃ in a second organic solvent at a second low temperature to yield the resulting organocerium reagent, and reacting the organocerium reagent at a third low temperature with a compound of Formula 1

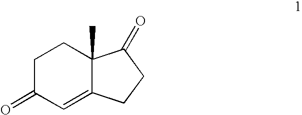

to yield the compound of Formula 2a.

In another embodiment, the invention encompasses the process of steps (a1) to (a5) above wherein the second organic solvent is selected from the group consisting of: tetrahydrofuran, 2-methyltetrahydrofuran, hexane, benzene, toluene, diethyl ether, chloroform, ethyl acetate and dichloromethane.

In another embodiment, the invention encompasses the process of steps (a1) to (a5) above wherein the second low temperature and third low temperature are independently about −70° C. to about −50° C.

In another embodiment, the invention encompasses the process of steps (a1) to (a5) above further comprising making TMS-acetylene-MgCl by (a6) reacting TMS-alkyne with iPrMgCl in a third organic solvent at a fourth low temperature to yield TMS-acetylene-MgCl.

In another embodiment, the invention encompasses the process of steps (a1) to (a6) above wherein the third organic solvent is selected from the group consisting of: tetrahydrofuran, 2-methyltetrahydrofuran, hexane, benzene, toluene, diethyl ether, chloroform, ethyl acetate and dichloromethane, and the fourth low temperature is about −5° C.

In another embodiment, the invention encompasses the process steps of (a1) to (a2), (a1) to (a4), (a1) to (a5) or (a1) to (a6) for making the compound of Formula I wherein A is F, B is H, C is H, D is F, E is H, F is a bond, G is —C(O)—N(R)(R) and each R is H.

In another embodiment, the invention encompasses the process of steps (a1) to (a2) for making the compound of Formula I wherein A is F, B is H, C is H, D is F, E is H, F is a bond, G is —C(O)—N(R)(R) and each R is H, further comprising making the compound of Formula 5, by (b1) reacting a compound of Formula 7

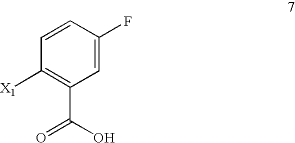

with a chlorinating agent in the presence of dimethylformamide in a fourth organic solvent to yield the acid chloride of Formula 7a

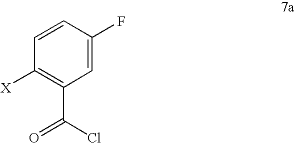

and (b2) reacting the acid chloride of Formula 7a with ammonium hydroxide to yield a compound of Formula 5.

In another embodiment, the invention encompasses the process of steps (a1) to (a2) and (b1) to (b2) above wherein the chlorinating agent is selected from the group consisting of: thionyl chloride, phosphorous pentachloride and oxalyl chloride.

In another embodiment, the invention encompasses the process of steps (a1) to (a2) and (b1) to (b2) above wherein the fourth organic solvent is selected from the group consisting of: tetrahydrofuran, 2-methyltetrahydrofuran, hexane, benzene, toluene, diethyl ether, chloroform, ethyl acetate and dichloromethane.

In another embodiment, the invention encompasses the hemihydrate of the compound of Formula Ia

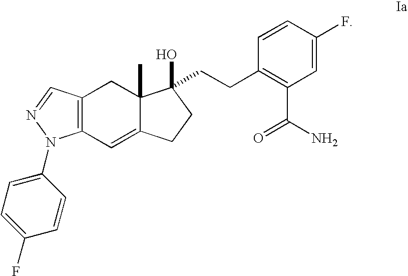

The terms “palladium catalyst” refers to a palladium complex capable of catalyzing the corresponding transformation. Examples of this complex include, but are not limited to [(allyl)PdCl]₂, palladium hydride, palladium on carbon, palladium(II) acetate, palladium(II) chloride, palladium (II) chloride acetonitrile complex, palladium (II) chloride benzonitrile complex, palladium(II) cyanide, palladium(II) nitrate, palladium(II) oxide, tetrakis(triphenylphosphine)palladium(0), tris(dibenzylideneacetone)dipalladium(0), and organopalladium complexes bearing phosphine ligands as defined below.

The term “phosphine ligand” refers to phosphine bearing one, two or three aliphatic or aromatic groups. Examples of this ligand include, but not limited to, (t-Bu)₃P.HBF₄, (t-Bu)₃P (tri-tertbutylphosphine), Ph₃P (triphenyl phosphine), (o-Tol)₃P (tri-ortho-tolylphosphine), Cy₃P (tricyclohexylphosphine), dppf (diphenylphosphinoferrocene), dppb (diphenylphosphinobutane), dppe (diphenylphosphinoethane), dppp (diphenylphosphinopropane), dppm (diphenylphosphinomethane) and di-tBu-2-(N-phenylpyrrole)phosphine.

The term “amine base” means for example primary, secondary, and tertiary amines, substituted amines including naturally occurring substituted amines, cyclic amines, for example, N,N-Diisopropylethylamine (Hünig's base), diethylamine, triethylamine, diisopropylamine and piperidine.

The term “metal catalyst” refers to single or combination of metal (s), metal complexes, or metal-impregnated solid support which are capable of performing the desired hydrogenation. Examples of this species include, but are not limited to, various weight % of palladium on carbon, various weight % of palladium hydroxide on carbon (Pearlman's catalyst) (wet or dry), palladium/platinum amalgam, rhodium on carbon, rhodium on alumina and platinum on carbon.

The term “polar aprotic solvent,” means, for example, 1,4-Dioxane, tetrahydrofuran, 2-methyltetrahydrofuran, acetone, acetonitrile, dimethylformamide, dimethyl sulfoxide, dimethoxymethane and 2-methyltetrahydrofuran.

The terms “first organic solvent,” “second organic solvent,” “third organic solvent,” and “fourth organic solvent” independently mean moderately polar aprotic and non-polar organic solvents, including, for example, tetrahydrofuran, 2-methyltetrahydrofuran, hexane, benzene, toluene, diethyl ether, chloroform, ethyl acetate and dichloromethane.

The terms “first elevated temperature” and “second elevated temperature” independently mean above about 50° C.

The terms “first low temperature,” “second low temperature,” “third low temperature,” and “fourth low temperature” independently mean below about 10° C.

The term “organic acid” means an organic compound with acidic properties, for example, acetic acid, formic acid, benzoic acid and p-toluenesulfonic acid.

The term “base” is well known in the art and means, for example, NaOH.

The term “chlorinating agent” means a reagent that reacts with a carboxylic acid to form an acid chloride, such as thionyl chloride, phosphorous pentachloride and oxalyl chloride.

The following abbreviations have the indicated meanings:
DIPEA=N,N′-diisopropylethylamine
Et=ethyl
DCE=dichloroethane
DMF=dimethylformamide
HATU=2-(1H-7-azabenzotriazol-1-yl)-1,1,3,3-tetramethyl uronium hexafluorophosphate methanaminium
Me=methyl
Ms=mesyl
MTBE=methyl t-butyl ether
NBS=N-bromosuccinimide
Ph=phenyl
TFA=trifluoroacetic acid
THF=tetrahydrofuran
TMEDA=tetramethylethylenediamine
TMS=trimethylsilyl

### EXAMPLES

### Example 1

### 1. Alkyne Addition

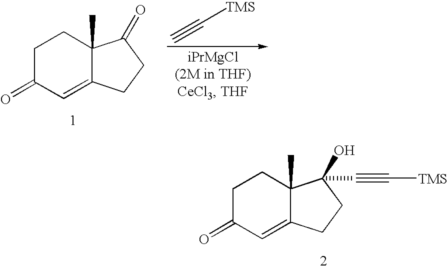

| substrate | MW | amount | mmol | equiv |
|---|---|---|---|---|
| 1 | 164 | 15 g | 91.46 | 1.0 |
| TMS alkyne | 98 | 13 g | 132.62 | 1.45 |
| iPrMgCl | | 71.14 mL | 128.05 | 1.4 |
| (1.8M in THF) | | | | |
| CeCl3 | 246 | 31.6 g | 128.05 | 1.4 |
| THF | | 50 + 150 + 45 mL | | |

To a round bottom flask with overhead stirring, N₂ inlet, thermocouple, and reflux condenser is added THF (150 mL) and anhydrous CeCl₃ and the resulting slurry was heated to 50° C. for 4 hr then 15 h at RT after which the flask is cooled to an internal temperature of −65° C. with a MeOH/dry ice bath.
Meanwhile, in a separate flask equipped with overhead stirring, N₂ inlet, and thermocouple was added THF (50 mL) and TMS alkyne and the resulting solution was cooled to an internal temperature of −5° C. iPrMgCl (1.8M in THF) is then added portionwise, while maintaining the internal temperature below 5° C. Once all the iPrMgCl is added (1.5 hr addition time), the reaction vessel is allowed to warm to room temperature and aged for 2 hr. After 2 hr, the newly formed alkyne-MgCl is cooled to 10° C. and added to the CeCl3 solution that has been previously cooled to −65° C., keeping the internal temperature below −50° C. Once all the alkyne-MgCl is added, the solution is aged for 1.5 hr at −60° C. Next, the ketone in THF (45 mL) is added via an addition funnel at −60° C. keeping the internal temperature below −50° C. Once all the ketone is added, the reaction is monitored with HPLC.

When the reaction is complete, as judged by HPLC conversion of 1, AcOH (2 mol equiv) is added (exothermic) at −50° C. and warmed to room temperature followed by addition of 30 mL of water.

The biphasic solution is then transferred to a 200 L extraction vessel containing water (30 mL) and MTBE (300 mL). After 20 min of agitation, the aqueous layer is cut and extracted with 100 mL of MTBE. The aqueous layer is cut again, checked for losses, and discarded. The combined organic layers are washed with 30 mL of fresh water then brine (30 mL), then concentrated and solvent switched to heptane to give the final composition of 1:15 of MTBE:heptane at 8-10 vol total. The resulting slurry is then aged at RT for overnight and filtered and the wetcake is washed with heptane and dried under a N₂ sweep. Isolated 18.5 g of the desired product (77% yield).

### 2. Pyrrazole Formation

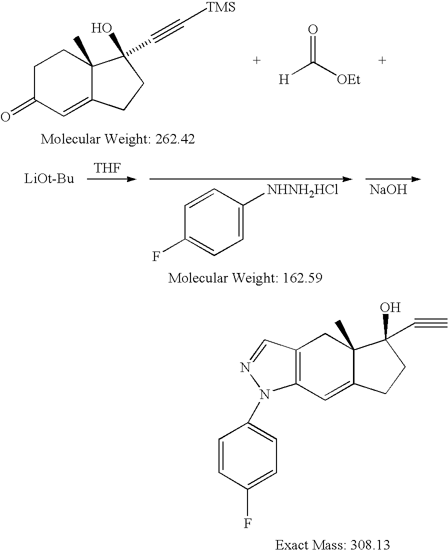

| Materials | MW | Amount | MMoles | Eq |
|---|---|---|---|---|
| Ketone SM | 260 | 11 g | 41.9 | 1.0 |
| Ethyl formate | 74 | 9.4 g | 127 | 3.0 |
| Li Ot-But | 80 | 17 g | 211 | 5.0 |
| THF | | 220 mL + 50 mL | | |
| AcOH | 60 | 25.4 g | 423 | 10 |
| MeOH | | 250 mL | | |
| p-F-phenylhydrazine | | | | |
| HCl salt | 162.6 | 8.24 g | 51 | 1.2 |

To a freshly prepared slurry of LitOBu in THF (220 mL) at 5° C. is added a solution of the enone and ethyl formate in 20 mL of THF over 10 min. After aging at 5-10° C. for 3 h, >95% conversion is typically observed, at which point a solution of AcOH in THF (25 mL) is added slowly over 10 min, while maintaining the temperature below 25° C. During this addition, solids form almost immediately and the batch thickens momentarily but becomes more fluid with stirring. At the end of AcOH quench, 25 mL of MeOH is then added, followed by p-F phenylhydrazine HCl salt as a solid. The reaction mixture is then heated to 60° C., aged for 1 h to give a full conversion, diluted with MTBE (110 mL) and washed with 10% aqueous NaCl (110 mL). The organic layer is separated and washed one more time with 10% aqueous NaCl (100 mL). Removal of the TMS group is carried out by first diluting the organic layer with 23 mL of MeOH and 23 mL of H₂O, followed by 42 mL of 10M NaOH to bring the pH to >13. After aging at 35-50° C. for 1-2 h, the reaction is found complete and the batch is cooled to 25° C., washed with 110 mL of 10% aqueous brine and the organic layer is washed one more time with 170 mL of 10% aqueous brine. The organic layer is then dried over Na₂SO₄ (20 g) overnight, filtered and then batch concentrated under vacuum to minimum volume (about 30 mL) using 160-200 mL of acetonitrile. Product crystallized out at this point and to this slurry is added 40 mL MTBE and then 450 L heptane over 30 min at. 23° C. After strirring for 35 min, reaction mixture is then concentrated under vacuum to remove about 20 mL of solvent. The batch is then stirred for 45 min, filtered and the wet cake is washed with 20 mL of 2:1 MTBE:heptane and air dried. The product is obtained as a brown solid in 9.1 grams (70%).

### 3. Coupling

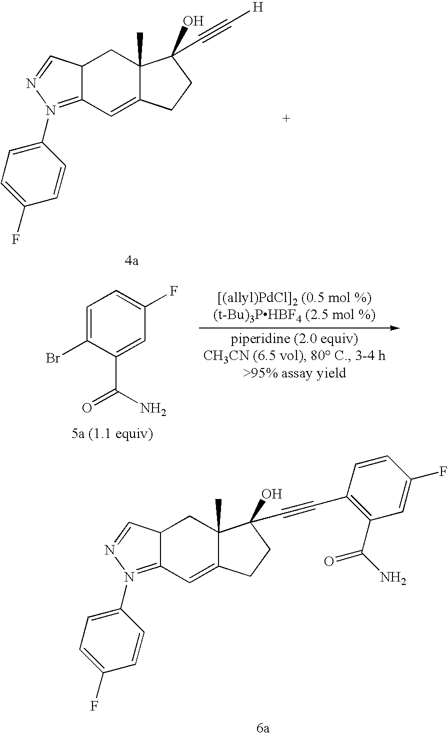

| Line | Reagent | FW | Amount | | mMoles |
|---|---|---|---|---|---|
| 1 | Alkyne 4a | 308.35 | 9.87 | gA | 32.0 |
| 2 | Bromide 5a | 218.02 | 7.67 | g | 35.2 |
| 3 | Piperdine | 85.15 | 6.39 | mL | 64.0 |
| 4 | [(allyl)PdCl]₂ | 365.89 | 58.8 | mgA | 0.160 |
| 5 | (t-Bu)₃P•HBF₄ | 290.13 | 232 | mgA | 0.800 |
| 6 | CH₃CN | 41.05 | 50 | mL | |
| 8 | Toluene | 92.14 | 100 | mL | |

Alkyne 4a, bromide 5a, acetonitrile (RM Table, line 6), and piperidine are charged successively to a round bottom flask equipped with a thermocouple, stir bar, and reflux condenser. The reagents are stirred until a reddish-brown solution is formed and the solution is degassed by 5 vacuum and nitrogen refill cycles. The phosphine ligand and palladium catalyst are then added successively and the resulting solution is degassed again. The solution is then heated to 80° C. and aged until a 99% conversion by HPLC analysis is achieved (typically 1 h). The solution is diluted with 100 mL of toluene and is then washed successively with HOAc (1.5 equiv) in 15 wt % aqueous NaCl (48 mL), saturated KHCO3 solution (40 mL), and saturated NaCl solution (40 mL). Ecosorb 941 (2.53 g) and trithiocyanuric acid (127 mg) are added to the solution and the solution was stirred between 23-25° C. for 1 hour. The black slurry is then filtered over Solka flock (10 g) through a 15-20 micron fritted funnel. The wet cake is washed with 130 mL of 2:1 toluene:CH₃CN. The solution is transferred to a separatory funnel and washed with 15 wt % K₂CO₃ aqueous solution (38 mL) and then diluted with toluene (26.7 mL) and CH₃CN (53 mL). The organic layer is washed with saturated aqueous NaCl (38 mL) and transferred to a round bottom flask. The organic layer is assayed to contain 12.76 gA of product 6a by HPLC analysis.

### 4. Crystallization of Coupling Product

The crude solution of 6a (12.6 g) in PhMe/MeCN is concentrated under reduced pressure to remove MeCN, while maintaining the total volume of 10 vol and the batch temperature at 20-25° C. Total of 6-vol of PhMe is used during this process. At the end of the solvent switch, the resulting slurry is heated up to 90° C. and cooled slowly to 72° C. After appropriate seeding, the product started to crystallize to give a slurry which is then aged overnight. Heptane (3.3 vol) is then added and the resulting mixture is aged until 6-8% of product remained in the mother liquor. At this point, the slurry is then filtered and the wetcake is washed with cold PhMe/Heptane (3/1, 6 vol) followed by heptane (3 vol) and dried under stream of N₂ overnight.
The product is isolated as pale yellow solid in 13.67 g (84.4 wt %) in 92% recovery or 81% overall yield.

### 5. Bromo Benzamide Preparation

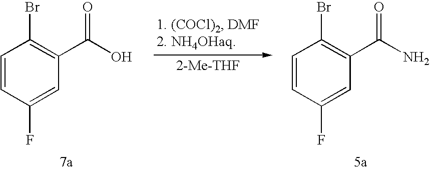

| reagents | Mw | amt. used | moles | equiv |
|---|---|---|---|---|
| 2-bormo-5- | 219.01 | 49.5 g | 226 | 1 |
| fluorobenzoic | | | | |
| acid | | | | |
| Oxalyl chloride | 126.93 | 21.4 mL | 248.6 | 1.1 |
| DMF | 73.09 | 0.871 mL | 11.3 | 0.05 |
| Ammonium | 35.05 | 62.6 mL | 927 | 4.1 |
| hydroxide | | | | |
| 2-Me-THF | | 250 mL | | |
| Water | | 10 L | | |
| 1 N HCl | | 5 L | | |
| Brine | | 10 L | | |
| PhMe | | 75 L + 12 L | | |
| Heptane | | 10 L + 7 L + 3 L | | |

To a RB flask equipped with an addition funnel is charged acid 7a, 2-Me-THF and DMF. The solution is then cooled to 7° C. and oxalyl chloride is added dropwise over 30 min at <15° C. After the addition is complete, the reaction mixture is warmed to rt and aged for 45 min. Upon complete consumption of the acid, the reaction mixture is then charged dropwise into another flask containing cold (9° C.) mixture of concentrated NH₄OH and 2-Me-THF over 1.5 h, while maintaining the temperature around 20-25° C. To the reaction mixture is added water (100 mL) to dissolve some solids and the resulting biphasic layer is transferred to a separatory funnel. The aqueous layer is separated and the organic layer is washed with 1 N HCl (50 mL) and with brine (100 mL). The final organic layer is then solvent switched to toluene to give a final slurry concentration of 15 vol. The slurry is then hheated to 110° C. to get a clear solution, which is then cooled slowly to RT. Crystallization is typically observed to occur at 100° C. and after aging at rt overnight, heptane (10 vol) is then added, followed by a 1 h of age. The suspension is then filtered and the wet cake is washed with cold 1:1 heptane:toluene and dried under a stream of N₂ to give the product in 46.9 g (94.7%).

### 6. Hydrogenation-Final Crystallization

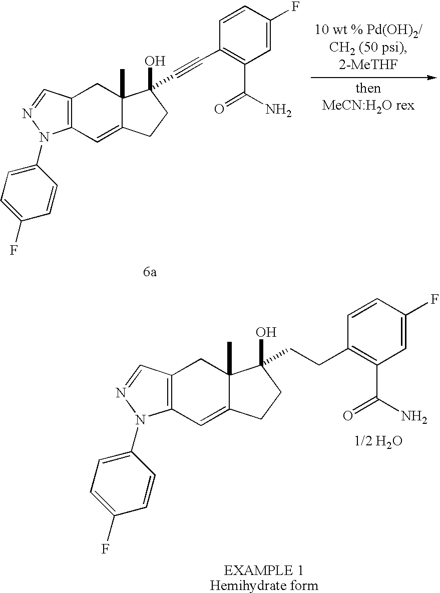

| COMPOUNDS | AMOUNT/MW | MMOL/EQ |
|---|---|---|
| Alkyne 6a | 4.86 g/445.46 | 10.91/1.0 |
| Wet 20%Pd(OH)₂/C | 56.9 g/140.43 | 0.56/0.06 |
| Hydrogen (H₂) | 1 atm | 21.82/2.0 |
| 2-MeTHF | 24 mL | 5 vol |
| THF | 24 mL | 5 vol |
| Solka Floc | 425 g | 75 wt % |
| Ecosorb C941 | 114 g | 20 wt % |
| MP-TMT | 46 g | 5 wt % |
| SiO₂ gel | 460 g | 50 wt % |
| MeCN | ~41-42 L | |
| H₂O | 26 L | |

A mixture of alkyne 6a and wet 20 wt % Pd(OH)₂/C in 2-MeTHF (5 vol) is exposed to 1 atm of H₂ for 6 hours, at which a complete consumption of starting is typically observed. The slurry is then diluted with THF (8 vol) and the resulting solution is filtered through Solka Floc (75 wt %) and rinsed with more THF (10 vol). The combined filtrate is filtered through a 1 micron inline filter into a round bottom flask and treated with 20 wt % Ecosorb C941 and 5 wt % MP-TMT and aged with rigorous stirring at 25° C. for 6 hours. The slurry is then filtered through 50 wt % SiO₂ gel, rinsed with 10 vol of THF and the combined filtrate is then solvent switched to MeCN to give a final slurry concentration of 13 vol. The slurry is then heated to 75° C., at which a clear yellowish solution is obtained, cooled to 72° C., seeded with 4% seeds and allowed to cool to 30° C. over 5-8 hours and aged for additional 8 hours. Water (8 vol) is then added over 3 hours, while maintaining the temperature between 28-30° C. At the end of addition, the resulting slurry is allowed to cool to 4° C. over 1-2 h, aged for additional 1 h, filtered and the wet cake is washed with cold 1:1 mixture of MeCN:H₂O. After drying at rt under a stream of N₂, 4.25 g of the product is isolated as white solid (87% yield).

### Reference Example 1

### Synthesis of 2-{2-[(4αS,5R)-1-(4-Fluorophenyl)-5-hydroxy-4α-methyl-1,4,4α,5,6,7-hexahydrocyclopenta[f]indazol-5-yl]ethyl}benzoic acid

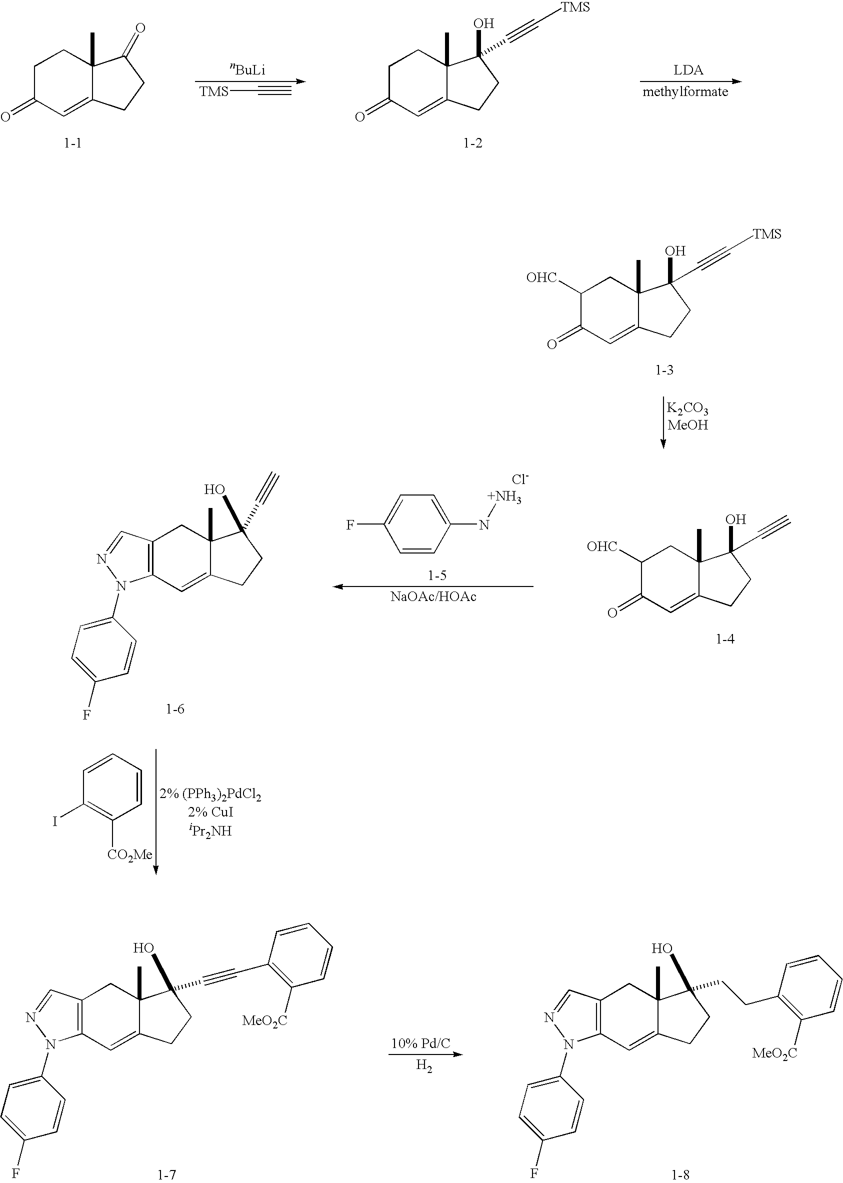

### (1S,7αS)-1-Hydroxy-7α-methyl-1-[(trimethylsilyl)ethynyl]-1,2,3,6,7,7α-hexahydro-5H-inden-5-one (1-2)

A 2.5M solution of ⁿBuLi (27.4 mL, 68.5 mmol) in hexanes was added dropwise to a solution of trimethylsilylacetylene (9.48 mL, 68.5 mmol) in THF (90 mL) at −78° C. The resulting solution was stirred at −78° C. for 30 min, then a solution of Hajos-Parrish Ketone (See Organic Syntheses, Coll. Vol. 7, p. 363; Vol 63, p. 26) (1-1, 7.5 g g, 45.7 mmol) in THF (90 mL) was added and the resulting solution stirred at −78° C. for 30 min. The reaction was quenched with saturated aqueous KH₂PO₄ and the crude product extracted with EtOAc (×3). The combined organic extracts were dried over anhydrous MgSO₄ and the solvent removed in vacuo. Purification by flash chromatography on 120 g of silica, eluting with a gradient of 0-55% EtOAc in hexanes afforded 9.54 g, 80% of 1-2 as a white solid.

MS (ESI): m/z=263.25 (MH⁺).

### (3S,3αS)-3-Hydroxy-3α-methyl-6-oxo-3-[(trimethylsilyl)ethynyl]-2,3,3α,4,5,6-hexahydro-1H-indene-5-carbaldehyde (1-3)

A 1.5 M solution of lithium diisopropylamide mono(tetrahydrofuran) in cyclohexane (121 mL, 182 mmol) was added to a solution of 1-2 (9.54 g, 36.4 mmol) in THF (400 mL) at −78° C. and the resulting solution stirred at this temperature for 1 hour to afford a thick suspension. Methyl formate (22.6 mL, 364 mmol) was added dropwise over about 15 min and the resulting suspension stirred at −78° C. for 5 hours. The reaction was quenched at −78° C. with 1 M aqueous HCl solution and the aqueous layer checked to ensure it was acidic. The crude product was extracted with EtOAc (×3) and the combined organic extracts were dried over anhydrous MgSO₄ and the solvent removed in vacuo to afford crude 1-3 (78% pure) that was used directly in the next step without purification.

MS (ESI): m/z=291.18 (MH⁺).

### (3R,3αS)-3-Ethynyl-3-hydroxy-3α-methyl-6-oxo-2,3,3α,4,5,6-hexahydro-1H-indene-5-carbaldehyde (1-5)

K₂CO₃ (5.03 g, 72.8 mmol) was added to a solution of crude 1-4 in MeOH (300 mL) and the resulting suspension stirred at ambient temperature for 90 min. The MeOH was removed in vacuo and 1M aqueous HCl was added to the residue and the crude product extracted with EtOAc (×3). The combined organic extracts were dried over anhydrous MgSO₄ and the solvent removed in vacuo. Purification by flash chromatography on 330 g of silica, eluting with a gradient of 0-70% EtOAc in hexanes afforded 5.94 g, 75% of 1-6 as a tan solid.

MS (ESI): m/z=219.25 (MH⁺).

### (4αS,5R)-5-Ethynyl-1-(4-fluorophenyl)-4α-methyl-1,4,4α,5,6,7-hexahydrocyclopenta[f]indazol-5-ol (1-6)

NaOAc (41.3 g, 504 mmol) was added to a solution of 1-5 (100 g, 458 mmol) and 4-fluorophenylhydrazine hydrochloride (1-5) (82 g, 504 mmol) in acetic acid (916 mL) and the resulting suspension stirred at ambient temperature for 1 hour. The reaction was quenched slowly (caution CO₂ evolution) with saturated aqueous NaHCO₃ solution and the crude product extracted with EtOAc (×3). The combined organic extracts were dried over anhydrous MgSO₄ and the solvent removed in vacuo. Purification by flash chromatography on 1.5 Kg of silica, eluting with a gradient of 0-100% EtOAc in hexanes afforded 133 g, 94% of 1-6 as a tan solid.

MS (ESI): m/z=309.2 (MH⁺).

### Methyl 2-{[(4αS,5R)-1-(4-fluorophenyl)-5-hydroxy-4α-methyl-1,4,4α,5,6,7-hexahydrocyclopenta[f]indazol-5-yl]ethynyl}benzoate (1-7)

Disopropylamine (2.85 mL, 20.0 mmol) was added to a solution of 1-6 (6.16 g, 20.0 mmol), methyl 2-iodobenzoate (6.28 g, 24.0 mmol), bis(triphenylphosphine)palladium (II) chloride (280 mg, 0.400 mmol), and CuI (76.0 mg, 0.400 mmol) in anhydrous THF (73 mL) at ambient temperature. The resulting solution was stirred at ambient temperature for 3.5 hours, then diluted with diethyl ether, filtered through a pad of celite and the solvent removed in vacuo. Purification by flash chromatography on 330 g of silica, eluting with a gradient of 0-90% EtOAc in hexanes afforded 8.47 g, 96% of 1-7 as an off white foamy solid.

MS (ESI): m/z=443.2 (MH⁺).

### Methyl 2-{2-[(4αS,5R)-1-(4-fluorophenyl)-5-hydroxy-4α-methyl-1,4,4α,5,6,7-hexahydrocyclopenta[f]indazol-5-yl]ethyl}benzoate (1-8)

10% Pd/C (8.16 g) was added to a solution of 1-7 (8.48 g, 19.2 mmol) in EtOAc (128 mL) at ambient temperature and the flask evacuated and backfilled with hydrogen. The resulting suspension was stirred at ambient temperature under a balloon of hydrogen for 45 min, filtered through a pad of celite and the solvent removed in vacuo to afford 7.92 g, 93% of 1-8 as a pale yellow solid.

MS (ESI): m/z=447.2 (MH⁺).

### Reference Example 2

### 5-fluoro-2-{2-[(4αS,5R)-1-(4-fluorophenyl)-5-hydroxy-4α-methyl-1,4,4α,5,6,7-hexahydrocyclopenta[f]indazol-5-yl]ethyl}benzamidE

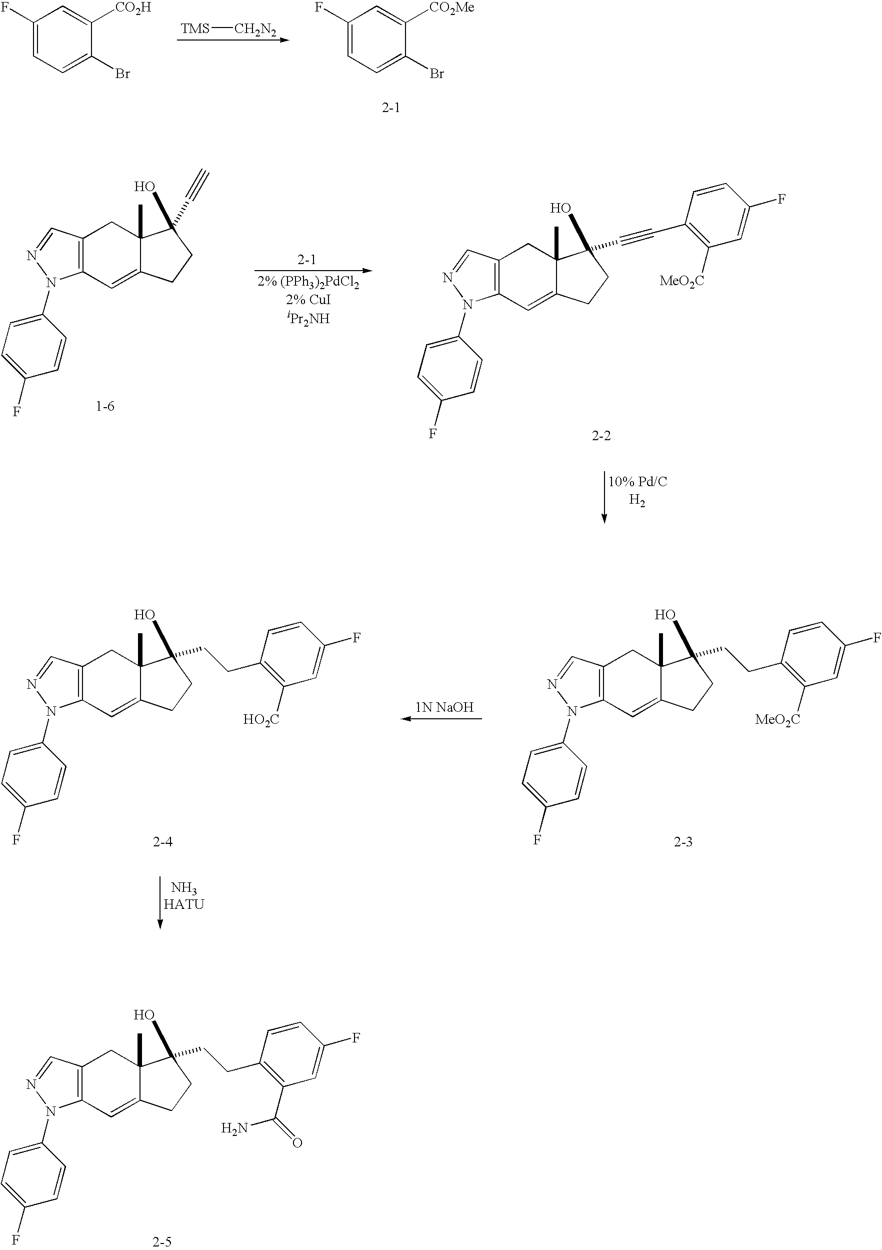

### Methyl 2-bromo-5-fluorobenzoate (2-1)

Trimethylsilyl diazomethane (338 ml, 676 mmol, 2.0 M in diethyl ether) was added dropwise to a stirred, 0° C. solution of 2-bromo-5-fluorobenzoic acid (74 g, 338 mmol) in MeOH (676 ml) until a yellow color persisted. Acetic acid was added dropwise until the yellow color dissipated. The solvent was removed in vacuo, and the resisdue was dissolved in CH₂Cl₂, then filtered through a plug of silica gel, eluting with CH₂Cl₂. The solvent was removed in vacuo to afford 77 g, 98% of 2-1 as a yellow oil.

### Methyl 5-fluoro-2-{[(4αS,5R)-1-(4-fluorophenyl)-5-hydroxy-4α-methyl-1,4,4α,5,6,7-hexahydrocyclopenta[f]indazol-5-yl]ethynyl}benzoate (2-2)

Diisopropylamine (14 ml, 97 mmol) was added to a solution of 1-6 (30 g, 97 mmol), 2-1 (27 g, 117 mmol), bis(triphenylphosphine)palladium (II) chloride (1.36 g, 1.95 mmol), and CuI (371 mg, 1.95 mmol) in anhydrous THF (354 ml) at ambient temperature. The resulting solution was stirred at 80° C. for 1 hour, then diluted with diethyl ether, filtered through a pad of celite and the solvent removed in vacuo. Purification by flash chromatography on 1.5 kg of silica, eluting with a gradient of 0-100% EtOAc in hexanes afforded 39 g, 86% of 2-2 as a white solid.

MS (ESI): m/z=461.33 (MH⁺).

### Methyl 5-fluoro-2-{2-[(4αS,5R)-1-(4-fluorophenyl)-5-hydroxy-4α-methyl-1,4,4α,5,6,7-hexahydrocyclopenta[f]indazol-5-yl]ethyl}benzoate (2-3)

10% Pd/C (17.9 g) was added to a solution of 2-2 (19.3 g, 42 mmol) in EtOAc (559 ml) at ambient temperature and the flask evacuated and backfilled with hydrogen. The resulting suspension was stirred at ambient temperature under a balloon of hydrogen for 1.5 hours, filtered through a pad of celite and the solvent removed in vacuo to afford 18.4 g, 94% of 2-3 as a white solid.

MS (ESI): m/z=465.37 (MH⁺).

### 5-Fluoro-2-{2-[(4αS,5R)-1-(4-fluorophenyl)-5-hydroxy-4α-methyl-1,4,4α,5,6,7-hexahydrocyclopenta[f]indazol-5-yl]ethyl}benzoic acid (2-4)

1M NaOH (151 ml, 151 mmol) was added to a solution of 2-3 (35 g, 75 mmol) in EtOH (300 ml) at ambient temperature. The solution was heated at 100° C. for 1 hour, acidified with 1N HCl and then extracted with EtOAc. The organic extract was washed with brine, dried over anhydrous MgSO₄, filtered and the solvent removed in vacuo to afford 37 g, 100% of 2-4 as a white solid.

MS (ESI): m/z=451.10 (MH⁺).

### 5-Fluoro-2-{2-[(4αS 5R)-1-(4-fluorophenyl)-5-hydroxy-4α-methyl-1,4,4α,5,6,7-hexahydrocyclopenta[f]indazol-5-yl]ethyl}benzamide (2-5)

A solution of ammonia in dioxane (0.5 M, 244 ml, 122 mmol), followed by HATU (31 g, 81 mmol) was added to a stirred solution of 2-4 (36.7 g, 81 mmol) and Hunigs base (43 ml, 244 mmol) in DMF (407 ml). The mixture was stirred for 1 hour, then was diluted with EtOAc and washed with sat NaHCO₃, brine, dried over anhydrous MgSO₄, filtered and the solvent was removed in vacuo. Purification by flash chromatography on 1.5 kg of silica, eluting with a gradient of 0-100% CHCl₃ to CHCl₃/EtOAc/MeOH (70:20:10) afforded 28 g, 76% of 2-5 as a white solid. The compound was dissolved in a minimal amount of boiling EtOAc, then allowed to cool slowly to ambient temperature to afford 14 g of crystalline material.

MS (ESI): m/z=450.1998 (MH⁺).

What is claimed is:

1. A process for synthesizing a compound of Formula I 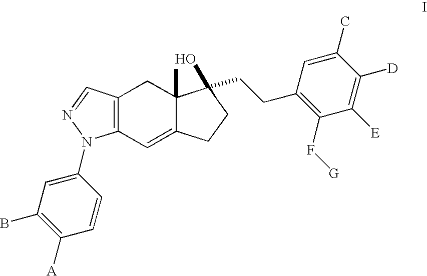
or a pharmaceutically acceptable salt thereof, wherein:
A and B are independently selected from the group consisting of: H, F and Cl;
C, D and E are independently selected from the group consisting of: H, F, Cl, —CN, —CH₃, —OCH₃, phenyl and —CF₃;
F is selected from the group consisting of: a bond, —C(R¹)(R²)— and —C(R¹)(R²)—C(R³)(R⁴)—;
G is selected from the group consisting of: —CN, —OH, —O—C(O)—N(R)(R), —O—C(O)—O—R, —C(O)—R, —C(O)—O—R, —NRR, aryl, substituted aryl, heteroaryl, substituted heteroaryl, —C(R^{a})(R^{b})—N(R)(R), —C(O)—N(R)(R), —C(O)—N(R)—C(R^{a})(R^{b})—R, —C(O)—N(R)—C(R^{a})(R^{b})—C(O)—OR, —C(O)—N(R)—C(R^{a})(R^{b})—C(O)—NRR, —N(R)—C(O)—R, —N(R)—C(O)—OR, —N(R)—C(O)—N(R)(R), —N(R)—S(O)ₙ—X, —S(O)n-N(R)(R), —N(R)—S(O)ₙ—N(R)(R) and —S(O)ₙ—X, wherein n is 0, 1 or 2;
each R is independently selected from the group consisting of: H, C₁₋₈alkyl, haloC₁₋₈alkyl, C₂₋₈alkenyl, haloC₂₋₈alkenyl, C₁₋₈alkoxy and C₃₋₆cycloalkyl-C₁₋₄alkyl-, and
two R groups attached to the same nitrogen atom can be joined together with the nitrogen atom to which they are attached to form a 3- to 7-membered monocyclic ring, said ring optionally substituted with oxo and said ring further optionally substituted with 1 to 3 substituents independently selected from the group consisting of: halo, hydroxyl, C₁₋₄alkyl and C₁₋₄alkoxy;
X is selected from the group consisting of: H, C₁₋₈alkyl, haloC₁₋₈alkyl, C₂₋₈alkenyl, haloC₂₋₈alkenyl, C₁₋₈alkoxy, C₃₋₆cycloalkyl, C₃₋₆cycloalkyl-C₁₋₄alkyl-, —CH₂—S(O)ₖ—CH₃, wherein k is 0, 1 or 2, aryl, substituted aryl, heteroaryl and substituted heteroaryl;
R¹, R², R³ and R⁴ are independently selected from the group consisting of: H, halo, C₁₋₄alkyl, hydroxy, C₃₋₆cycloalkyl and C₁₋₄haloalkyl, and R¹ and R² may be joined together with the carbon atom to which they are attached to form a 3- to 6-membered mono-cyclic ring;
R^{a} and R^{b} are independently selected from the group consisting of: H, C₁₋₄alkyl, C₁₋₄haloalkyl and hydroxy or R^{a} and R^{b} may be joined together with the carbon atom to which they are attached to form a 3- to 6-membered mono-cyclic ring; and
substituted aryl and substituted heteroaryl mean aryl and heteroaryl respectively, each substituted with one to three substituents independently selected from the group consisting of: halo, C₁₋₄alkyl, C₁₋₄haloalkyl and —CN;
comprising:
(a1) coupling a compound of Formula 4 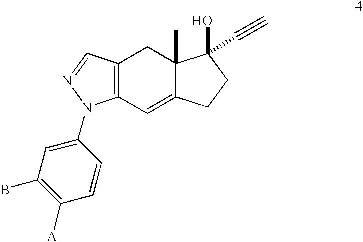
with a compound of Formula 5 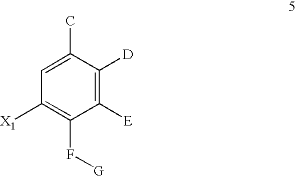
wherein X₁ is selected from the group consisting of: I, Br, Cl and OTf, in the presence of a palladium catalyst, a phosphine ligand and an amine base in a polar aprotic solvent at a first elevated temperature to yield a compound of Formula 6 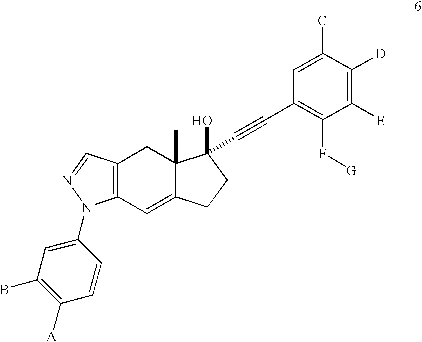
and (a2) hydrogenating the compound of Formula 6 with H₂ in the presence of a metal catalyst to yield the compound of Formula I;
and optionally converting the compound of Formula I into a pharmaceutically acceptable salt.

2. The process according to claim 1 wherein the palladium catalyst is selected from the group consisting of [(allyl)PdCl]₂, palladium hydride, palladium on carbon, palladium(II) acetate, palladium(II) chloride, palladium (II) chloride acetonitrile complex, palladium (II) chloride benzonitrile complex, palladium(II) cyanide, palladium(II) nitrate, palladium(II) oxide, tetrakis(triphenylphosphine)palladium(0), tris(dibenzylideneacetone)dipalladium(0), and organopalladium complexes bearing phosphine ligands.

3. The process according to claim 1 wherein the phosphine ligand is selected from the group consisting of: (t-Bu)₃P.HBF₄, tri-tertbutylphosphine, triphenyl phosphine, tri-ortho-tolylphosphine, tricyclohexylphosphine, diphenylphosphinoferrocene, diphenylphosphinobutane, diphenylphosphinoethane, diphenylphosphinopropane, diphenylphosphinomethane and di-tBu-2-(N-phenylpyrrole)phosphine.

4. The process according to claim 1 wherein the amine base is selected from the group consisting of: N,N-Diisopropylethylamine, diethylamine, triethylamine, diisopropylamine and piperidine.

5. The process according to claim 1 wherein the polar aprotic solvent is selected from the group consisting of: 1,4-Dioxane, tetrahydrofuran, 2-methyltetrahydrofuran, acetone, acetonitrile, dimethylformamide, dimethyl sulfoxide, dimethoxymethane and 2-methyltetrahydrofuran.

6. The process according to claim 1 wherein the first elevated temperature is about 80° C.

7. The process according to claim 1 wherein the metal catalyst is selected from the group consisting of: palladium on carbon, palladium hydroxide on carbon, palladium/platinum amalgam, rhodium on carbon, rhodium on alumina and platinum on carbon.

8. The compound according to claim 1 further comprising making the compound of Formula 4 by
(a3) reacting a compound of Formula 2a 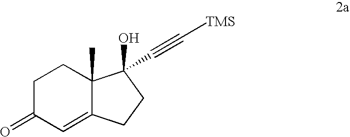
with H—C(O)—O—R, wherein R is C₁₋₄alkyl, in the presence of LiOtBu in a first organic solvent at a first low temperature to yield a compound of Formula 2b 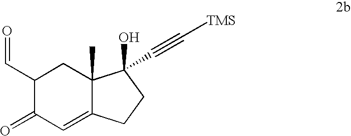
and (a4) quenching the reaction with an organic acid and, without further isolation, reacting the compound of Formula 2b with a compound of Formula 2c 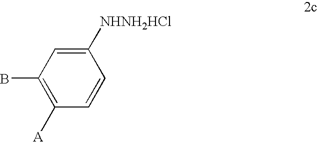
at a second elevated temperature and desilylating with a base, in either order, to yield a compound of Formula 4.

9. The process according to claim 8 wherein the first organic solvent is selected from the group consisting of: tetrahydrofuran, 2-methyltetrahydrofuran, hexane, benzene, toluene, diethyl ether, chloroform, ethyl acetate and dichloromethane.

10. The process according to claim 8 wherein the first low temperature is about 5° C. to about 10° C.

11. The process according to claim 8 wherein the organic acid is selected from the group consisting of: acetic acid, formic acid, benzoic acid and p-toluenesulfonic acid.

12. The process according to claim 8 wherein the second elevated temperature is about 60° C.

13. The process according to claim 8 wherein the base is sodium hydroxide.

14. The process according to claim 8 further comprising making the compound of Formula 2a by (a5) reacting TMS-acetylene-MgCl with CeCl₃ in a second organic solvent at a second low temperature to yield the resulting organocerium reagent, and reacting the organocerium reagent at a third low temperature with a compound of Formula 1 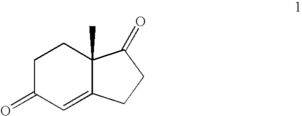
to yield the compound of Formula 2a.

15. The process according to claim 14 wherein the second organic solvent is selected from the group consisting of: tetrahydrofuran, 2-methyltetrahydrofuran, hexane, benzene, toluene, diethyl ether, chloroform, ethyl acetate and dichloromethane.

16. The process according to claim 14 wherein the second low temperature and the third low temperature are independently about −70° C. to about −50° C.

17. The process according to claim 14 further comprising making TMS-acetylene-MgCl by (a6) reacting TMS-alkyne with iPrMgCl in a third organic solvent at a fourth low temperature to yield TMS-acetylene-MgCl.

18. The process according to claim 17 wherein the third organic solvent is selected from the group consisting of: tetrahydrofuran, 2-methyltetrahydrofuran, hexane, benzene, toluene, diethyl ether, chloroform, ethyl acetate and dichloromethane, and the fourth low temperature is about −5° C.

19. The process according to claim 1 for making the compound of Formula I wherein A is F, B is H, C is H, D is F, E is H, F is a bond, G is —C(O)—N(R)(R) and each R is H.

20. The process according to claim 19 further comprising making the compound of Formula 5 by
(b1) reacting a compound of Formula 7 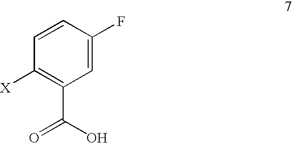
with a chlorinating agent in the presence of dimethylformamide in a fourth organic solvent to yield the acid chloride of Formula 7a 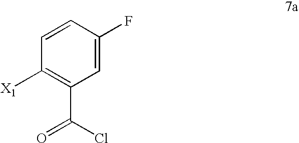
and (b2) reacting the acid chloride of Formula 7a with ammonium hydroxide to yield a compound of Formula 5.

21. The process according to claim 20 wherein the chlorinating agent is selected from the group consisting of: thionyl chloride, phosphorous pentachloride and oxalyl chloride.

22. The process according to claim 20 wherein the fourth organic solvent is selected from the group consisting of: tetrahydrofuran, 2-methyltetrahydrofuran, hexane, benzene, toluene, diethyl ether, chloroform, ethyl acetate and dichloromethane.

23. The hemihydrate of the compound of Formula Ia [chemical structure]
